(19) United States
(12) Patent Application Publication
(10) Pub. No.: US 2003/0191440 A1
(43) Pub. Date: Oct. 9, 2003

(54) Safety fluid collector

(76) Inventor: Edward Lichtenberg, Philadelphia, PA (US)

Correspondence Address: Edward Lichtenberg, 2401 Pennsylvania Ave. Apt. 18B27, Philadelphia, PA, 19130, US

(21) Appl. No.: 10/116,452

(22) Filed: Apr. 4, 2002

(51) Int. Cl. A61M 1/00

(52) U.S. Cl. 604/317; 600/576; 206/569

(57) ABSTRACT

A safety fluid collector uses a quick release device to allow a healthcare worker to release a used needle into a sharps container with one hand, and in a short time after blood samples have been obtained. The thumb of the healthcare worker is used to rotate a button on the needle holder which releases the needle into a sharps container. The sharps container comprises a rotating gate which allows needles enter the container but which blocks the exit of used needles if the container is accidentally tipped over.

### FIELD OF THE INVENTION

This invention involves a safety fluid collector. More specifically, it covers a low cost, easy to use blood sampling device which prevents accidental sticks of healthcare workers.

### BACKGROUND OF THE INVENTION

Every needle stick can lead to major health and cost problems to the healthcare worker, the hospital, and/or the patient. The most serious and prevalent problems occur as a result of blood drawing. Should a stick occur contaminated blood can cause hepatitis, HIV or other infectious diseases. Hence steps must be taken in blood drawing equipment design to significantly reduce the chance of an accidental stick.

### DESCRIPTION OF PRIOR ART

For many years the most commonly used blood-drawing device is supplied by Becton Dickinson Co. A double-ended needle is fastened to a holder which has male threads that engage female threads at one end of an open tube. The tube holds an evacuated vial (Vacutainer) which, is to receive the blood sample. One end of the hollow needle is positioned to puncture the Vacutainer cap. The other end punctures a vein to draw blood. After venipuncture a Vacutainer is inserted into the open end of the tube, causing blood to fill the Vacutainer. After blood drawing the healthcare worker removes the device and sets it down to attend to the patient. He then walks over to a sharps container where he can dispose of the needle-tube combination or he can unscrew the needle to dispose of it to reuse the Vacutainer tube.

This system allows an appreciable amount of time when the healthcare worker is exposed to accidental sticks. Proximity to the used needle enhances the risk. Unscrewing the needle for Vacutainer reuse also presents a chance for an accident. To simplify needle separation the manufacturer has designed a mechanism, part of the Vacutainer tube. However, the mechanism is relatively expensive. Many hospitals prohibit tube reuse because of possible contamination. An expensive needle separation device is therefore not practical.

Some needle-tube combinations are equipped with means (usually a plastic device) which is deployed by the healthcare worker immediately after removing the used needle. He disposes of the covered needle-tube after attending to the patient. The time of exposure to a used needle is reduced. However, the manipulation of the needle protector presents a chance for an accidental stick. Another disadvantage is the extra cost of the protector.

Another safety feature produced by Bio-Plexus consists of two needles, one inside the other. The internal needle is hollow and has a blunt end. After venipuncture by the sharp outer needle, the safety feature advances the blunt needle past the sharp tip of the outer needle. The needle is not removed from the patient until it is made safe. One problem with this system is that the sharp needle must have a large diameter. This makes entry into small veins more difficult. Also the use of this needle may not be advised for patients with thinned blood. Its high cost is a negative factor.

Finally, there are devices where the needle, after blood drawing and while still in the vein, is retracted by a spring into a protective tube and cannot cause an accidental stick. Reuse is not feasible, and the added cost may be a problem.

### SUMMARY OF THE INVENTION

The present invention overcomes the problems of prior art by its simplicity, which results in added safety against accidental sticks, lower cost of the device, lower disposal costs, and flexibility in use. This invention comprises a double ended needle in its holder with its protective covers and its connecting Vacutainer tube. It also includes a small sharps container which holds about 200 used needles. The forward needle enters a patient's vein. The rear needle punctures a Vacutainer cap when it is inserted to allow blood flow into the container. Before blood drawing has started the small sharps container is placed close to the patient.

The novelty of this invention involves the simple connection between the needle and tube. An L-shaped slot at the small diameter end of the tube is engaged by a button projecting from the side of the needle holder. To insert the needle the rear protective needle cover is removed. Using the forward needle cover as a tool the needle is inserted to slide the button in the axial section of the slot in the tube end, where it is a loose fit. The needle holder is then rotated through the radial leg of the slot, where it is a tight fit (about 0.007″ interference). At the end of the slot the button rests in a cavity that just fits its diameter. The needle is thus securely held to withstand blood drawing forces. After blood drawing the healthcare worker withdraws the needle, with his thumb on the holder button. He holds the needle vertically over the sharps container and rotates the button. The needle leaves the slot and drops into the sharps container. Needle withdrawal and disposal takes about one second, a minimum of time for an accidental stick.

With regard to reuse, button rotation in the slot has withstood 500 operations with no change in its function.

This invention has the following improvements in the state of the art.

1. Safety. Disposal of a used needle takes about one second, using one hand only. There is no need to provide a separate cover or an appreciable time for an exposed used needle. After the rapid disposal of the used needle the healthcare worker can concentrate his attention on the patient with no concern about an accidental stick.

2. Low Cost. The L-slot and the button are molded in the parts at no extra cost, Separate needle covers are not required. The small sharps container costs less than one cent per blood-drawing, much less than other safety devices.

3. Flexibility. Because of the low cost of the Vacutainer tube its reuse is not essential. However, normal slot operation can withstand many reuses.

### DESCRIPTION OF THE DRAWINGS

The advantages of this invention will be better understood by references to the following detailed description of the accompanying drawings wherein:

FIG. 1 is a sectional view of the safety fluid collector just after assembly of the needle holder into the Vacutainer tube.

FIG. 1A is along the line 1-1 of FIG. 1.

FIG. 1B is along the line 2-2 of FIG. 1.

FIG. 1C is a planar development of the L-slot.

FIG. 2 is a side view of the needle holder with both covers on.

FIG. 3 is a top view of the sharps container.

FIG. 3A is a section along the line 3-3 of FIG. 3.

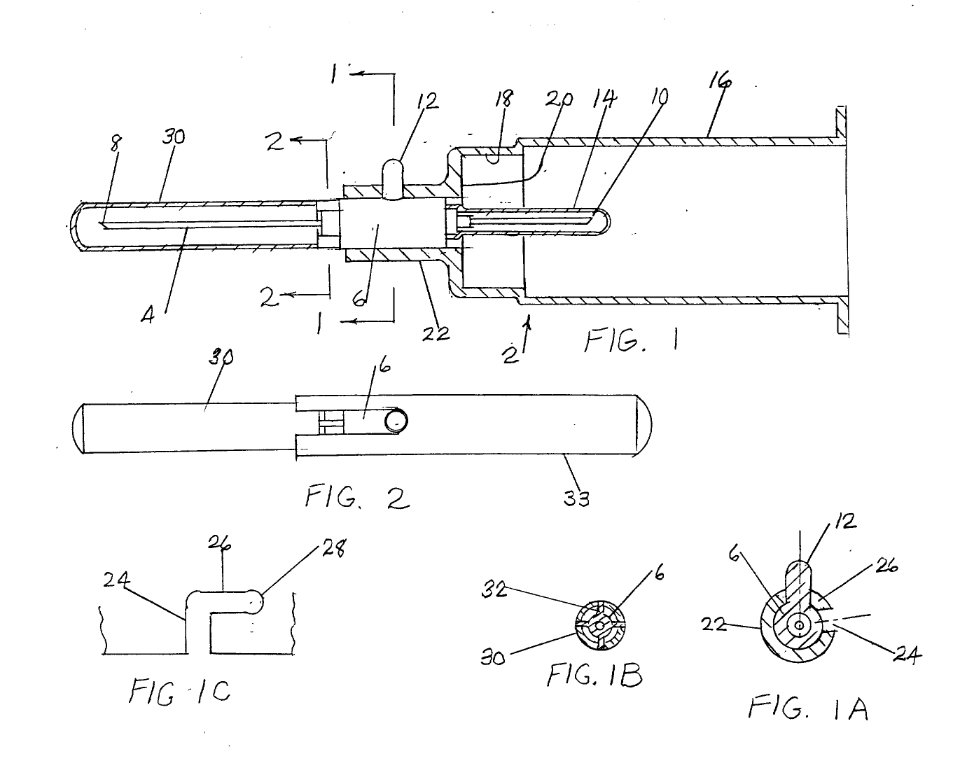

In FIG. 1 the fluid collector is shown in section. It comprises a double-ended hollow needle 4 fastened to a needle holder 6. The needle has two sharp ends: 8 for venipuncture and 10 for Vacutainer puncture. The needle holder 6 has a projecting button 12 whose purpose is described below. A flexible sheath covers needle 10 and is punctured by needle 10. This prevents blood flow when a Vacutainer has been filled and removed from needle 10 (present state of the art). A Vacutainer tube 16 is an open-ended tube which positions a Vacutainer (not shown) on diameter 18 and a shoulder 20. The end of the tube 16 has a small diameter projection 22 which contains an L-shaped slot. Button 12 fits into that slot, which is shown in planar development in FIG. 1C. The vertical (axial) section of the slot 24 is wider than the diameter of the button 12. The front portion of the horizontal (radial) section of slot 22 is a tight fit to button 12 so that it requires a torque to move the button in radial slot 26. At the end of radial slot 26 is a partial diameter 28 which just fits button 12. The healthcare worker places the needle holder 6 into the slot 22 by using the needle cover 30 as a tool until the button rests securely at the end 28 of the slot. He places the sharps container 101 near the patient, removes needle cover 30, and is ready for venipuncture. Vacutainers are inserted into tube 16, punctured and are filled with blood. Upon completion of blood collection the healthcare worker removes needle 8 while holding his thumb on button 12. He immediately places the needle, point down, over the sharps container 101, rotates the button 12, and the needle holder 6 drops into the sharps container. He then attends to the patient, with no chance of a needle stick.

FIG. 1A shows button 12 in its final position, and axial slot 24.

FIG. 1B shows four prongs 32, part of needle holder 6, which are engaged by four slots in needle cover 30, for use in assembly. 7

FIG. 2 shows the two needle covers 30 and 32 protecting the needles 8 and 10 before assembly starts. Needle cover 32 is removed just prior to start of assembly.

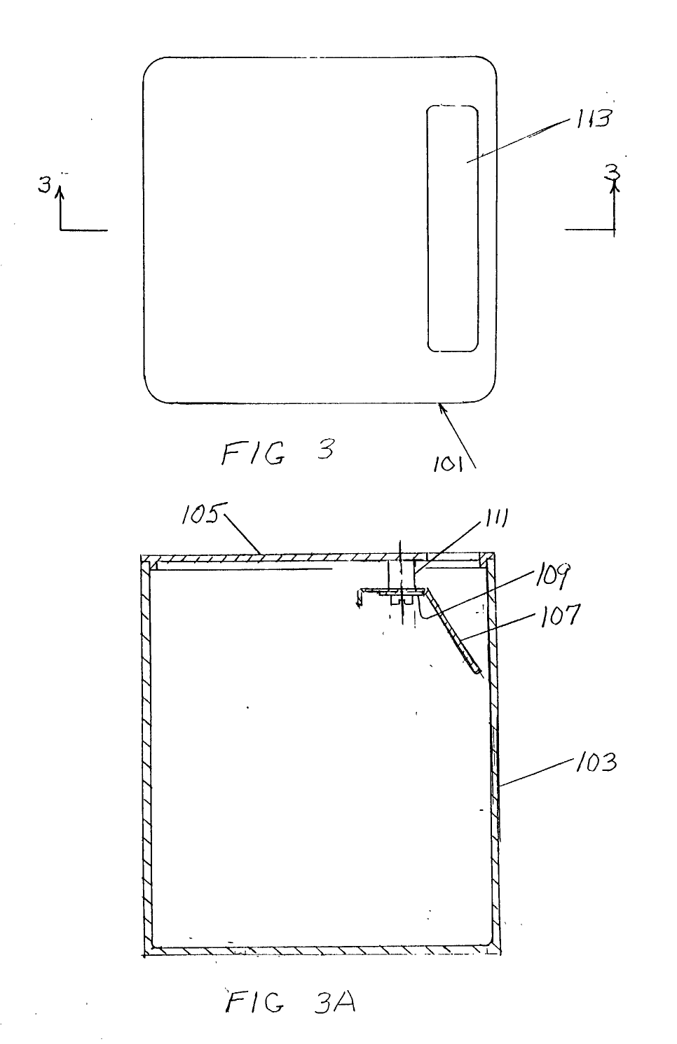

FIG. 3 is a top view of the sharps container, which holds about 200 used needles when full. It consists of an open container 103 and a lid which has a slot 113 through which used needles are discarded. The lid is permanently glued to container 103. A gate 107, seen in FIG. 3A, pivots about two spacers 109. The gate 107 rotates clockwise when a used needle strikes it as the needle is being discarded. This permits the needle to enter the container. The gate then swings back under the force of gravity to the position shown, thus preventing entrapped needles from leaving the container. Two tosses 111 project from the underside of lid 105 to which spacers 109 are fastened. Gate 107 normally rests on the spacers and is located by oversized holes in the gate. The holes surround bosses 111 and permit the gate to rotate. Should the container tip over, gate 107 moves by gravity to the inside surface of the lid 105. The gate is still positioned to entrap the needles.

I claim:

1. A safety fluid collector for drawing a blood sample from a patient, said safety fluid collector comprising:
A. A needle holder and a double-ended hollow needle having a forward section and a rearward section, projecting through said needle holder, further comprising an L-shaped slot, a rotatable button fastened to said needle holder and resting in the L-shaped slot, which is in the forward end of a Vacutainer holder, said button, when rotated, releases said button, needle holder and needle from said Vacutainer holder for disposal of said needle into a sharps container
B. A first needle cover covering said forward section of said hollow needle, said forward section being insertable into a patient for drawing blood
C. A second needle cover covering said rearward section of said hollow needle, said rearward section being inserted into a Vacutainer for collecting the blood and
D. A Vacutainer tube for holding the Vacutainer during blood drawing.

2. A sharps container for holding said used blood drawing needles and comprising:
A. A container with a top lid having an opening through which said used needles fall for holding and subsequent disposal thereof, and
B. A rotating gate mounted on the undersurface of said top lid, said gate rotating to allow the said needles to drop into said container for disposal, and said gate rotating into a blocking position if said container is tipped over, preventing the needles from falling out of said container.
